(19) United States
(12) Patent Application Publication
(10) Pub. No.: US 2006/0024702 A1
(43) Pub. Date: Feb. 2, 2006

(54) Device, system, and method for detecting a target molecule in a sample

(76) Inventors: Dennis M. Connolly, Rochester, NY (US); Jeffrey Hainon, Webster, NY (US); Scott N. Seabridge, Penfield, NY (US); Roberta J. Greco, Canandaigua, NY (US)

Correspondence Address: Integrated Nano-Technologies, LLC, 999 Lehigh Station Road, Henrietta, NY, 14467, US

(21) Appl. No.: 11/128,078

(22) Filed: May 12, 2005

Related U.S. Application Data
(60) Provisional application No. 60/570,231, filed on May 12, 2004.

(51) Int. Cl. C12Q 1/68; C12M 1/34

(52) U.S. Cl. 435/6; 435/287.2

(57) ABSTRACT

The present invention relates to a device suitable for detecting a target molecule in a sample using a detection card having two or more electrically separated conductors, wherein the sample can be analyzed for the presence of the target molecule by determining whether the conductors are electrically connected. The device has a platform for receiving the detection card and a lid moveable from a first position in engagement with the platform and a second position distal from the platform. The lid has a fluid interface suitable for establishing fluid communication between the device and the detection card and an electrical interface suitable for establishing electrical communication between the device and the detection card. Also disclosed is a system and a method for detecting a target molecule, both of which utilize the device.

This application claims the benefit of U.S. Provisional Patent Application Ser. No. 60/570,231, filed May 12, 2004, which is hereby incorporated by reference in its entirety.

### FIELD OF THE INVENTION

This invention relates to a device suitable for detecting a target molecule in a sample using a detection card, and systems and methods for detecting a target nucleic acid molecule in a sample using a detection card.

### BACKGROUND OF THE INVENTION

Nucleic acids, such as DNA or RNA, have become of increasing interest as analytes for clinical or forensic uses. Powerful new molecular biology technologies enable one to detect congenital or infectious diseases. These same technologies can characterize DNA for use in settling factual issues in legal proceedings, such as paternity suits and criminal prosecutions.

For the analysis and testing of nucleic acid molecules, amplification of a small amount of nucleic acid molecules, isolation of the amplified nucleic acid fragments, and other procedures are necessary. The science of amplifying small amounts of DNA have progressed rapidly and several methods now exist. These include linked linear amplification, ligation-based amplification, transcription-based amplification, and linear isothermal amplification. Linked linear amplification is described in detail in U.S. Pat. No. 6,027,923 to Wallace et al. Ligation-based amplification includes the ligation amplification reaction (LAR) described in detail in Wu et al., Genomics 4:560 (1989) and the ligase chain reaction described in European Patent No. 0320308B1. Transcription-based amplification methods are described in detail in U.S. Pat. No. 5,766,849 to McDonough et al., U.S. Pat. No. 5,654,142 to Kievits et al., Kwoh et al., Proc. Natl. Acad. Sci. USA 86:1173 (1989), and PCT Publication No. WO 88/10315 to Ginergeras et al. The more recent method of linear isothermal amplification is described in U.S. Pat. No. 6,251,639 to Kurn.

The most common method of amplifying DNA is by the polymerase chain reaction (“PCR”), described in detail by Mullis et al., Cold Spring Harbor Quant. Biol. 51:263-273 (1986), European Patent No. 201,184 to Mullis, U.S. Pat. No. 4,582,788 to Mullis et al., European Patent Nos. 50,424, 84,796, 258017, and 237362 to Erlich et al., and U.S. Pat. No. 4,683,194 to Saiki et al. The PCR reaction is based on multiple cycles of hybridization and nucleic acid synthesis and denaturation in which an extremely small number of nucleic acid molecules or fragments can be multiplied by several orders of magnitude to provide detectable amounts of material. One of ordinary skill in the art knows that the effectiveness and reproducibility of PCR amplification is dependent, in part, on the purity and amount of the DNA template. Certain molecules present in biological sources of nucleic acids are known to stop or inhibit PCR amplification (Belec et al., Muscle and Nerve 21(8):1064 (1998); Wiedbrauk et al., Journal of Clinical Microbiology 33(10):2643-6 (1995); Deneer and Knight, Clinical Chemistry 40(1):171-2 (1994)). For example, in whole blood, hemoglobin, lactoferrin, and immunoglobulin G are known to interfere with several DNA polymerases used to perform PCR reactions (Al-Soud and Radstrom, Journal of Clinical Microbiology 39(2):485-493 (2001); Al-Soud et al., Journal of Clinical Microbiology 38(1):345-50 (2000)). These inhibitory effects can be more or less overcome by the addition of certain protein agents, but these agents must be added in addition to the multiple components already used to perform the PCR. Thus, the removal or inactivation of such inhibitors is an important factor in amplifying DNA from select samples.

On the other hand, isolation and detection of particular nucleic acid molecules in a mixture requires a nucleic acid sequencer and fragment analyzer, in which gel electrophoresis and fluorescence detection are combined. Unfortunately, electrophoresis becomes very labor-intensive as the number of samples or test items increases.

For this reason, a simpler method of analysis using DNA oligonucleotide probes is becoming popular. New technology, called VLSIPS™, has enabled the production of chips smaller than a thumbnail where each chip contains hundreds of thousands or more different molecular probes. These techniques are described in U.S. Pat. No. 5,143,854 to Pirrung et al., PCT Publication No. WO 92/10092, and PCT WO 90/15070. These biological chips have molecular probes arranged in arrays where each probe ensemble is assigned a specific location. These molecular array chips have been produced in which each probe location has a center to center distance measured on the micron scale. Use of these array type chips has the advantage that only a small amount of sample is required, and a diverse number of probe sequences can be used simultaneously. Array chips have been useful in a number of different types of scientific applications, including measuring gene expression levels, identification of single nucleotide polymorphisms, and molecular diagnostics and sequencing as described in U.S. Pat. No. 5,143,854 to Pirrung et al.

Array chips where the probes are nucleic acid molecules have been increasingly useful for detection of the presence of specific DNA sequences. Most technologies related to array chips involve the coupling of a probe of known sequence to a substrate that can either be structural or conductive in nature. Structural types of array chips usually involve providing a platform where probe molecules can be constructed base by base or by covalently binding a completed molecule. Typical array chips involve amplification of the target nucleic acid followed by detection with a fluorescent label to determine whether target nucleic acid molecules hybridize with any of the oligonucleotide probes on the chip. After exposing the array to a sample containing target nucleic acid molecules under selected test conditions, scanning devices can examine each location in the array and quantitate the amount of hybridized material at that location. Alternatively, conductive types of array chips contain probe sequences linked to conductive materials such as metals. Hybridization of a target nucleic acid typically elicits an electrical signal that is carried to the conductive electrode and then analyzed.

For most solid support or array technologies, small oligonucleotide capture probes are immobilized or synthesized on the support. The sequence of the capture probes imparts the specificity for the hybridization reaction. Several different chemical compositions exist currently for capture probe studies. The standard for many years has been straight deoxyribonucleic acids. The advantage of these short single stranded DNA molecules is that the technology has existed for many years and the synthesis reaction is relatively inexpensive. Furthermore, a large body of technical studies is available for quick reference for a variety of scientific techniques, including hybridization. However, many different types of DNA analogs are now being synthesized commercially that have advantages over DNA oligonucleotides for hybridization. Some of these include PNA (protein nucleic acid), LNA (locked nucleic acid) and methyl phosphonate chemistries. In general, all of the DNA analogs have higher melting temperatures than standard DNA oligonucleotides and can more easily distinguish between a fully complementary and single base mis-match target. This is possible because the DNA analogs do not have a negatively charged backbone, as is the case with standard DNA. This allows for the incoming strand of target DNA to bind tighter to the DNA analog because only one strand is negatively charged. The most studied of these analogs for hybridization techniques is the PNA analog, which is composed of a protein backbone with substituted nucleobases for the amino acid side chains (see www.appliedbiosystems.com or www.eurogentec.com). Indeed, PNAs have been used in place of standard DNA for almost all molecular biology techniques including DNA sequencing (Arlinghaus et al., Anal Chem. 69:3747-53 (1997)), DNA fingerprinting (Guerasimova et al., Biotechniques 31:490-495 (2001)), diagnostic biochips (Prix et al., Clin. Chem. 48:428-35 (2002); Feriotto et al., Lab Invest 81:1415-1427 (2001)), and hybridization based microarray analysis (Weiler et al., Nucleic Acids Res. 25:2792-2799 (1997); Igloi, Genomics 74:402-407 (2001)).

Techniques for forming sequences on a substrate are known. For example, the sequences may be formed according to the techniques disclosed in U.S. Pat. No. 5,143,854 to Pirrung et al., PCT Publication No. WO 92/10092, or U.S. Pat. No. 5,571,639 to Hubbell et al. Although there are several references on the attachment of biologically useful molecules to electrically insulating surfaces such as glass (http://www.piercenet.com/Technical/default.cfm?tmpl=../Lib/ViewDoc.cfm&doc=3483; McGovern et al., Langmuir 10:3607-3614 (1994)) or silicon oxide (Examples 4-6 of U.S. Pat. No. 6,159,695 to McGovern et al.), there are few examples of effective molecular attachment to electrically conducting surfaces except for gold (Bain et al., Langmuir 5:723-727 (1989)) and silver (Xia et al., Langmuir 22:269, (1998)). In general, the problem of attaching biologically active molecules to the surface of a substrate, whether it is a metal electrical conductor or an electrical insulator such as glass, is more difficult than the simple chemical reaction of a reactive group on the biological molecule with a complementary reactive group on the substrate. For example, a metal electrical conductor has no reactive sites, in principle, except those that may be adventitiously or deliberately positioned on the surface of the metal.

Hybridization of target DNAs to such surface bound capture probes poses difficulties not seen, if both species are soluble. Steric effects result from the solid support itself and from too high of a probe density. Studies have shown that hybridization efficiency can be altered by the insertion of a linker moiety that raises the complementary region of the probe away from the surface (Schepinov et al., Nucleic Acid Res. 25:1155-1161 (1997); Day et al., Biochem J. 278:735-740 (1991)), the density at which probes are deposited (Peterson et al., Nucleic Acids Res. 29:5163-5168 (2001); Wilkins et al., Nucleic Acids Res. 27:1719-1729)), and probe conformation (Riccelli et al., Nucleic Acids Res. 29:996-1004 (2001)). Insertion of a linker moiety between the complementary region of a probe and its attachment point can increase hybridization efficiency and optimal hybridization efficiency has been reported for linkers between 30 and 60 atoms in length. Likewise, studies of probe density suggest that there is an optimum probe density, and that this density is less than the total saturation of the surface (Schepinov et al., Nucleic Acid Res. 25:1155-1161 (1997); Peterson et al., Nucleic Acids Res. 29:5163-5168 (2001); Steel et al., Anal. Chem. 70:4670-4677 (1998)). For example, Peterson et al. reported that hybridization efficiency decreased from 95% to 15% with probe densities of 2.0×10¹² molecules/cm² and 12.0×10¹² molecules/cm², respectively.

Quantitation of hybridization events often depends on the type of signal generated from the hybridization reaction. The most common analysis technique is fluorescent emission from several different types of dyes and fluorophores. However, quantitating samples in this manner usually requires a large amount of the signaling molecule to be present to generate enough emission to be quantitated accurately. More importantly, quantitation of fluorescence generally requires expensive analysis equipment for linear response. Furthermore, the hybridization reactions take up to two hours, which for many uses, such as detecting biological warfare agents, is simply too long. Therefore, a need exists for a system which can rapidly detect and quantitate biological material in samples.

The present invention is directed to achieving these objectives.

### SUMMARY OF THE INVENTION

One aspect of the present invention relates to a device suitable for detecting a target molecule in a sample using a detection card having two or more electrically separated conductors. The sample can be analyzed for the presence of the target molecule by determining whether the conductors are electrically connected. The device has a platform for receiving the detection card and a lid moveable from a first position in engagement with the platform to a second position distal from the platform. The lid has a fluid interface suitable for establishing fluid communication between the device and the detection card when the lid is in the first position, but not the second position, and the detection card is positioned on the platform. The lid also has an electrical interface suitable for establishing electrical communication between the device and the detection card when the lid is in the first position, but not the second position, and the detection card is positioned on the platform.

Another aspect of the present invention relates to a system for detecting a target molecule in a sample. This system includes a detection card having one or more pairs of electrical conductors, each pair including a first electrical conductor and a second electrical conductor, where the electrical conductors are not in contact with one another. The detection card also has one or more sets of two oligonucleotide probes attached to the electrical conductors. The probes are positioned such that they cannot come into contact with one another and such that a target nucleic acid molecule, which has two sequences, a first sequence complementary to a first probe attached to the first electrical conductor and a second sequence complementary to a second probe attached to the second electrical conductor, can bind to both probes. The system also includes a device suitable for receiving the detection card. The device has a platform for receiving the detection card and a lid moveable from a first position in engagement with the platform to a second position distal from the platform. The lid has a fluid interface suitable for establishing fluid communication between the device and the detection card when the lid is in the first position, but not the second position, and the detection card is positioned on the platform. The lid also has an electrical interface suitable for establishing electrical communication between the device and the detection card when the lid is in the first position, but not the second position, and the detection card is positioned on the platform.

A further aspect of the present invention relates to a method of detecting a target molecule in a sample. This method involves providing a detection system including a detection card having one or more pairs of electrical conductors, each pair including a first electrical conductor and a second electrical conductor, where the electrical conductors are not in contact with one another. The detection card also has one or more sets of two oligonucleotide probes attached to the electrical conductors. The probes are positioned such that they cannot come into contact with one another and such that a target nucleic acid molecule, which has two sequences, a first sequence complementary to a first probe attached to the first electrical conductor and a second sequence complementary to a second probe attached to the second electrical conductor, can bind to both probes. The detection card also has a sample injection port through which a sample can be introduced into the detection card. The system further includes a device suitable for receiving the detection card. The device has a platform for receiving the detection card and a lid moveable from a first position in engagement with the platform to a second position distal from the platform. The lid has a fluid interface suitable for establishing fluid communication between the device and the detection card when the lid is in the first position, but not the second position, and the detection card is positioned on the platform. The lid also has an electrical interface suitable for establishing electrical communication between the device and the detection card when the lid is in the first position, but not the second position, and the detection card is positioned on the platform. The method further involves injecting a sample, potentially containing the target molecule, into the sample injection port. The detection card is positioned on the platform with the lid in the second position. The lid is moved into the first position such that fluid communication and electrical communication is established between the device and the detection card by the fluid interface and the electrical interface, respectively. The sample is processed within the detection card under conditions effective to permit any of the target molecule present in the sample to bind to the capture probes and thereby connect the capture probes. The presence of the target molecule is detected by determining whether electricity is conducted between the electrically separated conductors.

### BRIEF DESCRIPTION OF THE DRAWINGS

FIG. 1 is a perspective view of the detection device of the present invention in the second position, with a detection card positioned on the detection device.

FIGS. 2A-D are side views of the detection device of FIG. 1. In FIG. 2A the lid is positioned in the first position in engagement with the platform such that fluid communication is established between the fluid interface of the device and a detection card positioned on the platform and electrical communication is established between the electrical interface of the device and the detection card positioned on the platform. In FIG. 2B, the lid is positioned in the second position distal from the platform. FIG. 2C shows the connection made between the fluid interface and a detection card positioned on the platform when the lid is in the first position. FIG. 2D shows the connection made between the electrical interface and a detection card positioned on the platform when the lid is in the first position.

FIG. 3 is a perspective view of the detection system of the present invention which includes a detection device and a detection card positioned on the detection device.

FIG. 4 is a schematic view of the fluidic system connected to the detection device of the present invention.

FIG. 5 is a top view of a detection card suitable for use in accordance with the present invention. The dashed lines represent fluid pathways and a detection chip which reside underneath the top surface of the detection card.

FIG. 6 depicts a single test structure on a detection chip suitable to be positioned in the detection reservoir of the detection card of the present invention. Oligonucleotide probes are attached to electrical conductors in the form of spaced apart conductive fingers.

FIG. 7 shows how a target nucleic acid molecule present in a sample is detected by the detection chip of the present invention.

### DETAILED DESCRIPTION OF THE INVENTION

One aspect of the present invention relates to a device suitable for detecting a target molecule in a sample using a detection card having two or more electrically separated conductors. The sample can be analyzed for the presence of the target molecule by, determining whether the conductors are electrically connected. The device has a platform for receiving the detection card and a lid or cover moveable from a first position in engagement with the platform to a second position distal from the platform. The lid has a fluid interface suitable for establishing fluid communication between the device and the detection card when the lid is in the first position, but not the second position, and the detection card is positioned on the platform. The lid also has an electrical interface suitable for establishing electrical communication between the device and the detection card when the lid is in the first position, but not the second position, and the detection card is positioned on the platform.

Another aspect of the present invention relates to a system for detecting a target molecule in a sample. This system includes a detection card having one or more pairs of electrical conductors, each pair including a first electrical conductor and a second electrical conductor, where the electrical conductors are not in contact with one another. The detection card also has one or more sets of two oligonucleotide probes attached to the electrical conductors. The probes are positioned such that they cannot come into contact with one another and such that a target nucleic acid molecule, which has two sequences, a first sequence complementary to a first probe attached to the first electrical conductor and a second sequence complementary to a second probe attached to the second electrical conductor, can bind to both probes. The system also includes a device suitable for receiving the detection card. The device has a platform for receiving the detection card and a lid moveable from a first position in engagement with the platform to a second position distal from the platform. The lid has a fluid interface suitable for establishing fluid communication between the device and the detection card when the lid is in the first position, but not the second position, and the detection card is positioned on the platform. The lid also has an electrical interface suitable for establishing electrical communication between the device and the detection card when the lid is in the first position, but not the second position, and the detection card is positioned on the platform.

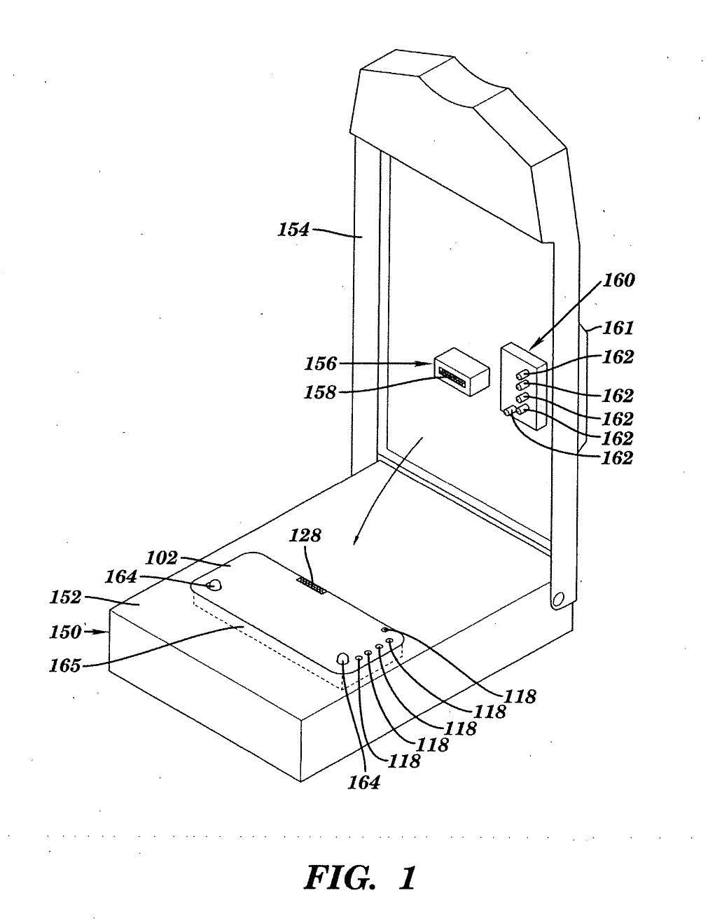

FIG. 1 is a perspective view of the device of the present invention. Device 150 has platform 152 and lid 154. Platform 152 is equipped with alignment pins 164 which correspond to holes in detection card 102 to ensure proper orientation of the detection card on platform 152. Platform 152 also may optionally include heat exchanger 165 such that when detection card 102 is positioned on platform 152, fluid temperatures in detection card 102 can be controlled. Lid 154 of device 150 is equipped with fluid interface 160, which has injectors 162, and electrical interface 156, which has electrical contacts 158. Cover 161 is mounted on lid 154 above fluid interface 160 and electrical interface 156.

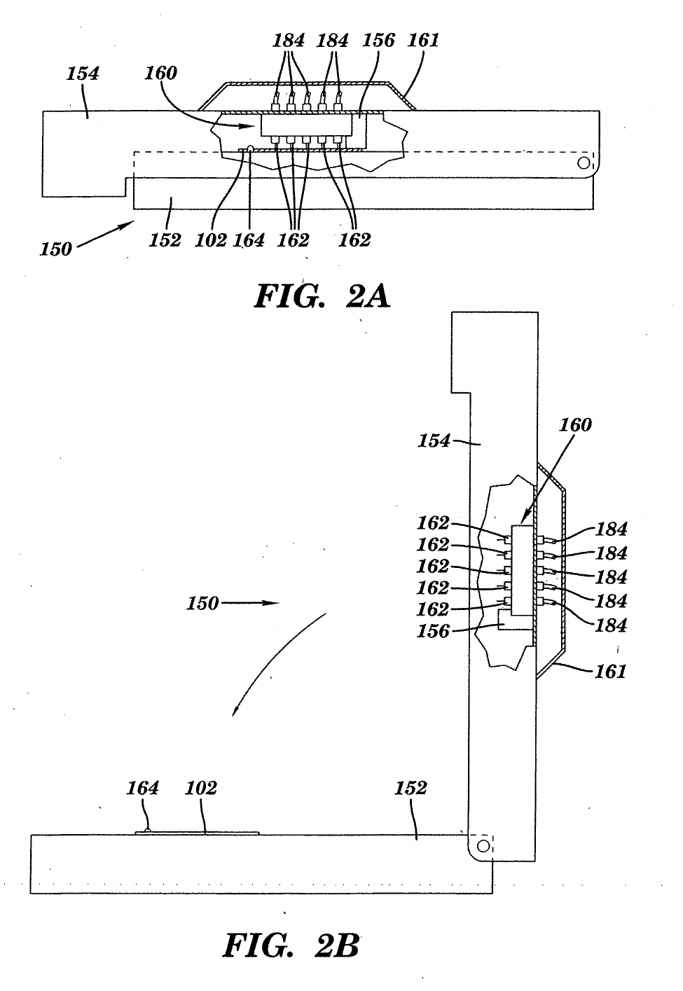

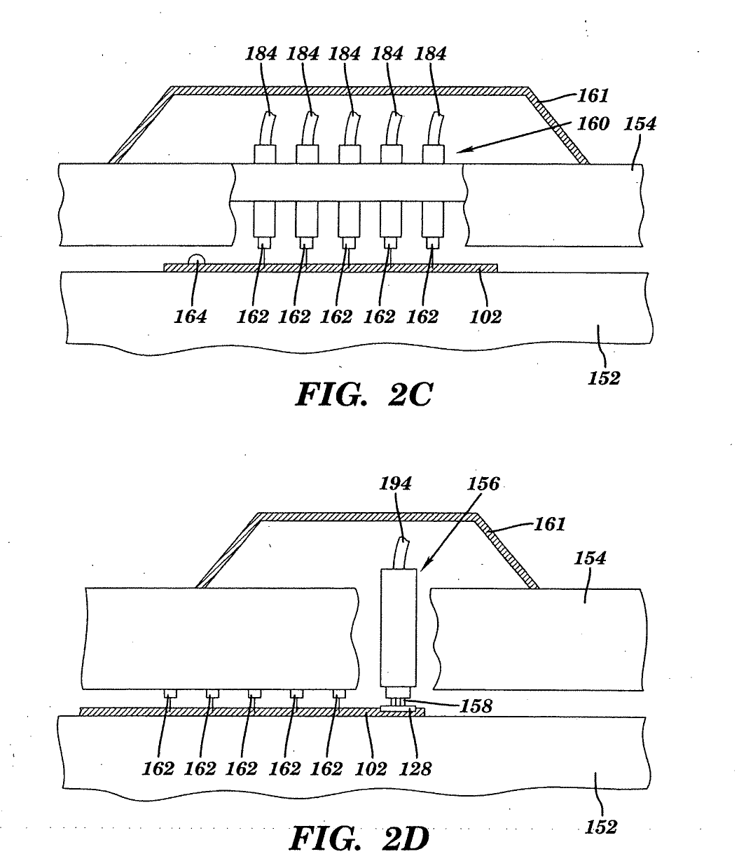

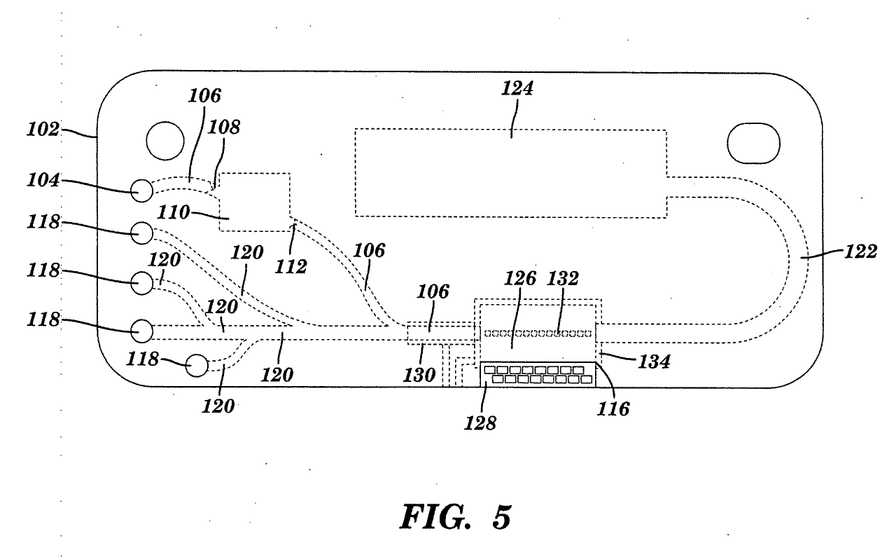

As shown in FIGS. 2A-B, lid 154 is moveable from a first position in engagement with platform 152 (FIG. 2A) to a second position distal from platform 152 (FIG. 2B). Preferably, lid 154 is pivotally moveable between the first and second positions. Fluid communication is established between device 150 and detection card 102 when injectors 162 come into contact with fluid injection ports 118 (FIG. 5) by moving lid 154 into the first position, as shown in more detail in FIG. 2C. By moving lid 154 into the first position, electrical contacts 158 and card contacts 128 also come into contact, thereby establishing electrical communication between device 150 and detection card 102. This is shown in more detail in FIG. 2D.

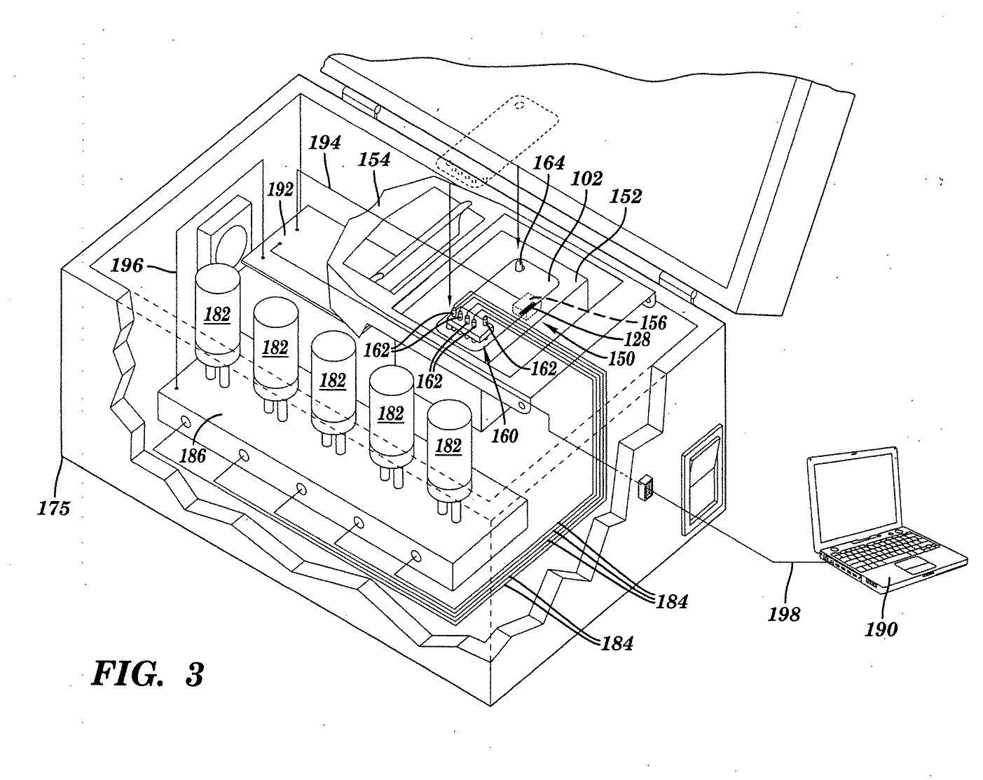

Device 150 also has a fluidic system connected to fluid interface 160, a controller electrically connected to electrical interface 156, and an external computer electrically coupled to the controller, all of which are illustrated in FIG. 3, and described in greater detail infra.

FIG. 3 shows a perspective view of the system of the present invention, which is organized in container 175. The system includes device 150, as described supra, and detection card 102 positioned on platform 152 of device 150. As shown in FIG. 3, lid 154 is in the first position in engagement with platform 152.

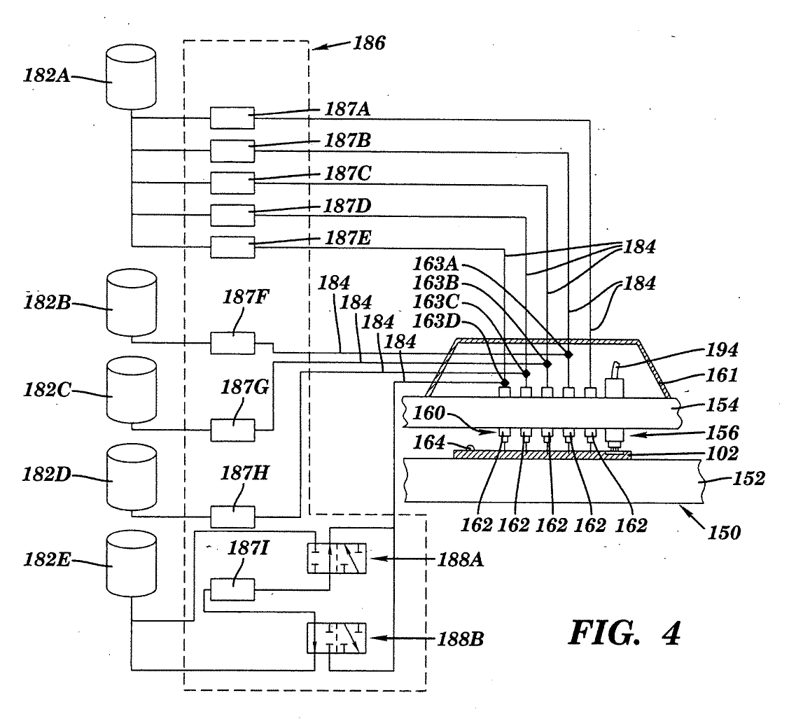

Connected to electrical interface 156 is controller 192. Controller 192 is connected to electrical interface 156 by electrical connector 194. Digital coupling 198 connects controller 192 to computer 190 so that device 150 can be operated using computer control. Controller 192 receives power from an external power connection. A main control board of controller 192 handles all serial communications to and from computer 190 (via electrical coupling 198) and to and from other control boards, which are also part of controller 192. The main control board also performs all of the auto scale resistor measurements of the test structures on detection chip 126 of detection card 102, by receiving signals from electrical interface 156 via electrical connector 194. A pump control board that controls all of the pumping operations of fluid displacement device 186, is also part of controller 192. The pump control board is connected to fluid displacement device 186 by electrical connector 196. As shown in FIG. 4, the pump control board provides control signals to pumps 187 and solenoid valves 188 so that fluids are properly dispensed from vials 182 to fluid interface 160, and eventually, into detection reservoir 116 of detection card 102. Thus, the introduction of fluids into detection card 102 is controlled from computer 190, by sending serial data through electrical coupling 198, which reaches controller 192. Controller 192 may optionally include a thermoelectric cooler control board that controls the automated temperature control of device 150, which includes providing heat to heat exchanger 165.

Connected to fluid interface 160 is a fluidic system, which includes vials 182, fluid lines 184, and fluid displacement device 186. Vials 182 contain fluid, which includes, without limitation, water, neutralizers, buffers, conductive ion solutions, enhancers, and other reagents which aid in the hybridization and/or metallization of nucleic acid molecules. The contents of vials 182 can be replenished. This is achieved by making vials 182 sealed and disposable, or by making them refillable. Fluid lines 184 carry fluid from vials 182 into fluid displacement device 186, and eventually, into injectors 162 of fluid interface 160. Fluid displacement device 186 carries out the function of forcing fluid from vials 182 through fluid lines 184 into fluid interface 160.

As illustrated in one embodiment in FIG. 4, fluid displacement device 186 (represented by dashed lines) is a multi-channeled manifold connected to a plurality of pumps and, optionally, one or more solenoid valves. As shown in FIG. 4, pumps 187A-I draw fluid from fluid vials 182A-E into fluid displacement device 186 and out through fluid lines 184 to injectors 162 of fluid interface 160. In the particular embodiment illustrated in FIG. 4, vial 182A contains water and pumps 187A-E draw water from fluid vial 182A into five separate fluid lines 184, each of which connects to a single injector 162. Vials 182B-E contain reagents which aid in the hybridization and/or metallization of nucleic acid molecules, including metal solution, a first developer solution, a second developer solution, and a buffer solution, respectively. The reagents contained in vials 182B-E are drawn into fluid displacement device 186 through fluid lines 184 by pumps 187F-I, respectively. As discussed in more detail infra, the fluids and solutions contained in vials 182A-E are introduced into detection card 102 and, eventually, reach detection reservoir 116 where they aid in the detection of the target molecule.

As illustrated in FIG. 4, a single injector 162 may receive more than one fluid line 184 by employing “Y” fittings 163A-D. When employed, a “Y” fitting preferably receives one fluid line carrying water and a second fluid line carrying a reagent. Thus, “Y” fittings 163 serve the function of permitting either water from one fluid line, or a reagent from a second fluid line, to enter a single injector 162. In operation of the particular embodiment illustrated in FIG. 4, pumps 187F-I are activated, in turn, to deliver reagents from vials 182B-E to fluid interface 160 to test for the presence of a target molecule in detection card 102. After testing is complete, pumps 187B-E are activated to deliver water from vial 182A to fluid interface 160, by passing through “Y” fittings 163A-D. Water forced through injector 162 after the reagents serves the function of washing out reagent residues and preventing the build-up of precipitates. The particular embodiment illustrated in FIG. 4 is also suitable for forcing reagents from a detection card containing reservoirs with reagents, as described in more detail infra.

The particular embodiment of fluid displacement device 186 illustrated in FIG. 4 also includes two-way solenoid valves 188. Solenoid valves 188 are optionally employed in the fluidic system of the detection device of the present invention to aid in the detection of a target molecule in the detection card. Solenoid valves 188A-B are connected to vial 182E and pump 187I by fluid lines 184. Fluid lines 184 also connect solenoid valves 188A-B to a injector 162 of fluid interface 160. In operation, solenoid valves 188A-B modulate the flow of buffer solution from vial 182E into fluid displacement device 160, and eventually, detection reservoir 116 of detection card 102. By modulating the flow of buffer solution in detection reservoir 116, a sample potentially containing the target molecule is moved back and forth across the electrical conductors of detection chip 126 to improve the chance of hybridization of the target molecule to the capture probes.

A detection card for use in the system of the present invention is illustrated in detail in FIG. 5. Detection card 102 has a fluid pathway (represented by dashed lines in FIG. 5) which resides under the exterior surface of the card. A sample is introduced into the fluid pathway through first injection port 104. Channel 106 connects first injection port 104 to detection reservoir 116. Archival reservoir 110 is positioned in channel 106 between first injection port 104 and detection reservoir 116. Waste reservoir 124 is connected to detection reservoir 116 by channel 122. Reagents may be introduced into the fluid pathway through injection ports 118. Channels 120 connect injection ports 118 to channel 106 and, eventually, to detection reservoir 116. The fluid pathway of detection card 102 also includes one-way valves 108 and 112 which permit fluid to flow only in one direction (i.e., towards detection reservoir 116).

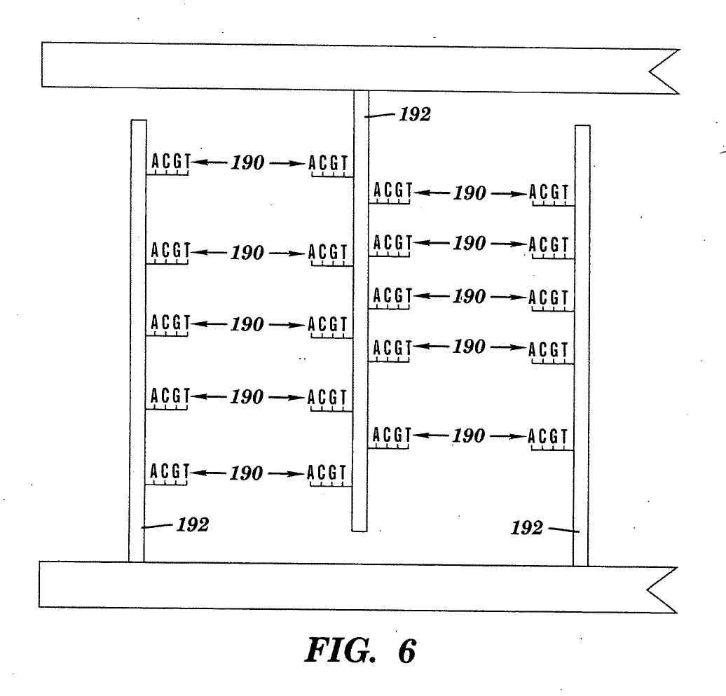

Detection card 102 also includes detection chip 126, and, optionally, heating element 134 and DNA concentrator 130. Detection chip 126 has one or more pairs of electrical conductors, each pair including a first electrical conductor and a second electrical conductor and one or more sets of two oligonucleotide probes attached to the electrical conductors. FIG. 6 depicts a single test structure of detection chip 126. According to FIG. 6, oligonucleotide probes 190, attached to spaced apart electrical conductors 192, are physically located at a distance sufficient that they cannot come into contact with one another. Detection chip 126, including the electrical conductors and attached oligonucleotide probes, reside at least partially within detection reservoir 116. Card contacts 128 of detection chip 126 are exposed to the exterior surface of detection card 102. Card contacts 128 are electrically connected to the conductors, such that electrical contacts 158 make contact with card contacts 128 when lid 154 is in the first position and detection card 102 is positioned on platform 152. Optional heating element 134 is located near detection chip 126 and enables fluid temperatures in detection card 102, particularly detection reservoir 116, to be controlled (in the event device 150 does not have a heating element). DNA concentrator 130 is located at the portion of channel 106 nearest detection reservoir 116. As described more fully in U.S. Provisional Patent Application Ser. No. 60/470,645 (which is hereby incorporated by reference in its entirety), the DNA concentrator causes DNA in detection reservoir 116 to move closer to detection chip 126 by application of an electrode having a polarity which electrostatically attracts target molecules in a flowing fluid sample.

Other detection cards suitable for use in conjunction with the system of the present invention are described in greater detail in the patent application entitled, “Detection Card for Analyzing a Sample for a Target Nucleic Acid Molecule, and Uses Thereof,” filed May 12, 2004, with express mail certificate number EL 984956968 US, which is hereby incorporated by reference in its entirety.

The system of the present invention is used for the detection of nucleic acid sequences from a sample. This involves a sample collection method whereby bacteria, viruses, or other DNA containing species are collected and concentrated. The system also incorporates a sample preparation method that involves the liberation of the genetic components. After liberating the nucleic acid, the sample is injected into a detection card which includes a detection chip containing complementary nucleic acid probes for the target of interest. In this manner, the detection chip may contain multiple sets of probe molecules that each recognizes a single but different nucleic acid sequence. This process ultimately involves the detection of hybridization products.

In the collection phase, bacteria, viruses, or other DNA containing samples are collected and concentrated. A plurality of collection methods will be used depending on the type of sample to be analyzed. Liquid samples will be collected by placing a constant volume of the liquid into a lysis buffer. Airborne samples can be collected by passing air over a filter for a constant time. The filter will be washed with lysis buffer. Alternatively, the filter can be placed directly into the lysis buffer. Waterborne samples can be collected by passing a constant amount of water over a filter. The filter can then be washed with lysis buffer or soaked directly in the lysis buffer. Dry samples can be directly deposited into lysis buffer for removal of the organism of interest.

After sample collection and lysis, cell debris can be removed by precipitation or filtration. Ideally, the sample will be concentrated by filtration, which is more rapid and does not require special reagents. Samples will be forced through filters that will allow only the cellular material to pass through, trapping whole organisms and broken cell debris.

A further aspect of the present invention relates to a method of detecting a target molecule in a sample. This method involves providing a detection system including a detection card having one or more pairs of electrical conductors, each pair including a first electrical conductor and a second electrical conductor, where the electrical conductors are not in contact with one another. The detection card also has one or more sets of two oligonucleotide probes attached to the electrical conductors. The probes are positioned such that they cannot come into contact with one another and such that a target nucleic acid molecule, which has two sequences, a first sequence complementary to a first probe attached to the first electrical conductor and a second sequence complementary to a second probe attached to the second electrical conductor, can bind to both probes. The detection card also has a sample injection port through which a sample can be introduced into the detection card. The system further includes a device suitable for receiving the detection card. The device has a platform for receiving the detection card and a lid moveable from a first position in engagement with the platform to a second position distal from the platform. The lid has a fluid interface suitable for establishing fluid communication between the device and the detection card when the lid is in the first position, but not the second position, and the detection card is positioned on the platform. The lid also has an electrical interface suitable for establishing electrical communication between the device and the detection card when the lid is in the first position, but not the second position, and the detection card is positioned on the platform. The method further involves injecting a sample, potentially containing the target molecule, into the sample injection port. The detection card is positioned on the platform with the lid in the second position (either before or after the sample is injected into the sample injection port). The lid is moved into the first position such that fluid communication and electrical communication is established between the device and the detection card by the fluid interface and the electrical interface, respectively. The sample is processed within the detection card under conditions effective to permit any of the target molecule present in the sample to bind to the capture probes and thereby connect the capture probes. The presence of the target molecule is detected by determining whether electricity is conducted between the electrically separated conductors.

The detection of a target molecule using the system of the present invention, can be carried out as follows. After lysis and clarification of the sample, the sample is introduced into detection card 102 through first injection port 104. The sample passes through channel 106 and into detection reservoir 116. Before or after the sample is introduced, detection card 102 is positioned on platform 152 of device 150 with lid 154 being in the second position distal from platform 152. After detection card 102 is positioned on platform 152 and the sample is introduced into detection card 102, lid 154 is moved to the first position in engagement with platform 152 such that injectors 162 of fluid interface 160 are in fluid communication with fluid injection ports 118 (FIG. 2C), and electrical contacts 158 are in electrical communication with detection chip 126 (FIG. 2D).

The sample is processed in detection reservoir 116 for a period of time sufficient for detection of a target nucleic acid molecule in the sample. Processing of the sample within detection reservoir 116 can involve neutralizing the sample, contacting the neutralized sample with a buffer, contacting the sample with metal ions, then contacting the sample with a metal deposition solution. Processing a sample within detection reservoir 116 involves the movement of reagents from vials 182 into fluid interface 160 through fluid lines 184. Movement of the reagents is achieved by fluid displacement device 186 as described supra. Alternatively, reagents may be held in reservoirs inside the detection card, as described in the patent application entitled “Detection Card for Analyzing a Sample for a Target Nucleic Acid Molecule, and Uses Thereof,” filed May 12, 2004 with express mail certificate number EL 984956968 US, which is hereby incorporated by reference in its entirety. When reservoirs inside the detection card are employed, fluid displacement device 186 can force a material, such as air, water, or oil, through fluid lines 184 into fluid interface 160 and into injection ports 118 to force the reagents from the reservoirs into channels which lead to detection reservoir 116. The embodiment illustrated in FIG. 4 is especially adapted to forcing a material from a reservoir contained in a detection card into a channel leading to detection reservoir 116. In particular, pumps 187A-E are connected to vial 182A which may contain air, water, or oil suitable for forcing a reagent from a reservoir contained in a detection card. Thus, when desired, pumps 187A-E may be activated to draw the forcing material from vial 182A into fluid lines 184 and, eventually, to injectors 162. The forcing material then enters detection card 102 at ports 118 and forces reagents from reservoirs within detection card 102 into detection reservoir 116. Water may then be introduced into vial 182A and forced through fluid lines 184 and injectors 162 to eliminate the build-up of precipitates.

Processing may further involve oscillating fluid back and forth in detection reservoir 116 of detection card 102 to increase binding efficiency of the target molecule in the sample to the oligonucleotide probes. Oscillation of fluid within detection reservoir 116 may be achieved by engaging one or more solenoid valves of the fluidic system as described supra. Molecules that are not captured by the oligonucleotide probes are expelled from detection reservoir 116 through channel 122 and into waste reservoir 124.

Processing of the sample within detection card 102 can be controlled by data communicated from computer 190 to controller 192 by way of digital coupling 198. Electrical connector 196 places fluid displacement device 186 in electrical contact with controller 192 so that data reaching controller 192 from computer 190 can be relayed to fluid displacement device 186.

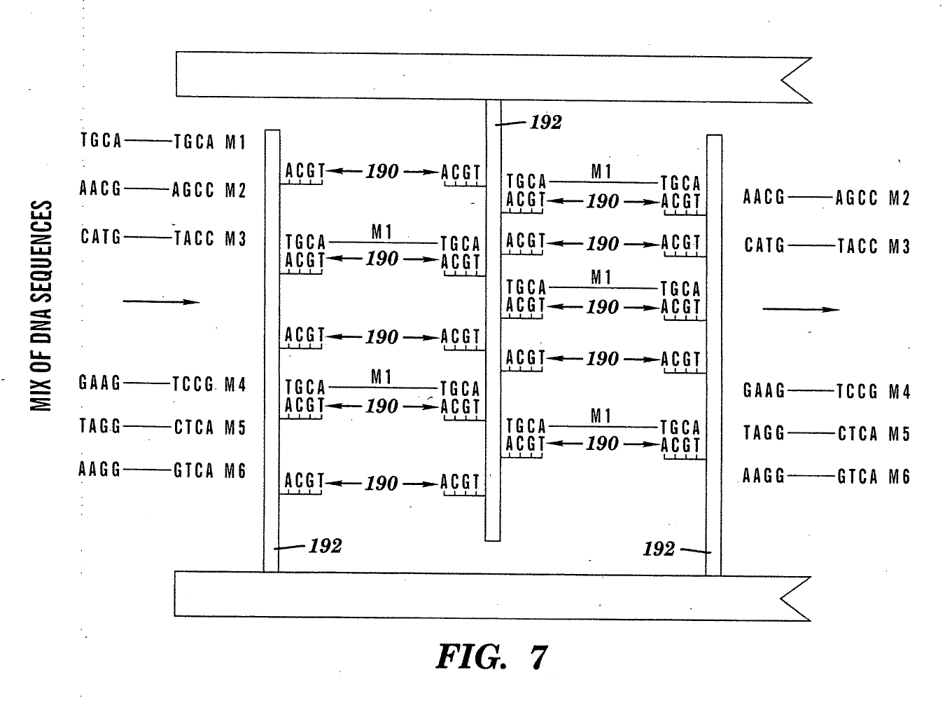

Binding of a target molecule to the oligonucleotide probes positioned on detection chip 126 to enable detection of the target in the sample is illustrated in FIG. 7. A sample, containing a mixture of nucleic acid molecules (i.e., M1-M6) to be tested, is discharged into detection card 102 and moved into detection reservoir 116 where it comes into contact with detection chip 126, including electrical conductors 192 and oligonucleotide probes 190. If a target nucleic acid molecule (i.e., M1) that is capable of binding to two oligonucleotide probes 190 is present in the sample, the target nucleic acid molecule will bind probes 190. If bound, the nucleic acid molecule can bridge the gap between two electrical conductors 192 and provide an electrical connection which can be detected by electrical interface 156 when lid 154 is in the first position. Signal of an electrical connection is thus passed from detection chip 126 to electrical interface 156 which relays the signal to controller 192 and, eventually, to computer 190 where it can be analyzed on a visual display. Any unhybridized nucleic acid molecules (i.e., M2-M6) not captured by the probes is washed away and passed into waste reservoir 124 of detection card 102.

The electrical connection provided by the target nucleic acid molecule arises from the electrical conductivity of nucleic acid molecules. Hans-Werner Fink and Christian Schoenenberger reported in Nature 398:407-410 (1999), which is hereby incorporated by reference in its entirety, that DNA conducts electricity like a semiconductor. This flow of current can be sufficient to construct a simple switch, which will indicate whether or not a target nucleic acid molecule is present within a sample. The presence of a target molecule can be detected as an “on” switch, while a set of probes not connected by a target molecule would be an “off” switch. The information can be processed by a digital computer which correlates the status of the switch with the presence of a particular target. The information can be quickly identified to the user as indicating the presence or absence of the biological material, organism, mutation, or other target of interest.

In a preferred embodiment of the present invention, after the target molecules have hybridized to sets of biological probes, the target molecule is contacted with metal ions under conditions effective to bind the metal ions on one or more sites of the target molecule. The target molecule with bound metal ions on one or more of its sites is then contacted with a metal under conditions effective to deposit metal on the target molecules hybridized to the probes as described in the U.S. patent application Ser. No. 10/763,597, which is hereby incorporated by reference in its entirety. Alternatively, metal particles may be mordanted on one or more sites of the nucleic acid molecule and metal deposited upon the mordanted nucleic acid molecule as described in U.S. Patent Application Ser. No. 60/533,342, which is hereby incorporated by reference in its entirety. The target nucleic acid molecule can then conduct electricity across the gap between the pair of probes. As described supra, this flow of current can be sufficient to construct a simple switch, which will indicate whether or not a target nucleic acid molecule is present within a sample.

Contacting the target molecules with metal ions can be carried out by moving fluid from the vials of the device through fluid lines leading to the fluid interface as described supra. The metal ions are then injected into the detection card through the fluid injection ports, whereupon the metal ions pass into the detection reservoir and bind target molecules.

The detection chip, on which conductive fingers 192 are fixed, is constructed on a support. Examples of useful support materials include, e.g., glass, quartz, and silicon as well as polymeric substrates, e.g. plastics. In the case of conductive or semi-conductive supports, it will generally be desirable to include an insulating layer on the support. However, any solid support which has a non-conductive surface may be used to construct the device. The support surface need not be flat. In fact, the support may be on the walls of a chamber in a chip.

The detection card described in this invention can be as simple as a device recognizing a single DNA sequence and hence a single organism, or as complex as recognizing multiple DNA sequences. Therefore, different types of detection cards can be constructed depending on the complexity of the application.

In carrying out the method of the present invention, a sample collection phase is initially carried out where bacteria, viruses, or other species are collected and concentrated. The target nucleic acid molecule whose sequence is to be determined is usually isolated from a tissue sample. If the target nucleic acid molecule is genomic, the sample may be from any tissue (except exclusively red blood cells). For example, whole blood, peripheral blood lymphocytes or peripheral blood mononuclear cells (“PBMC”), skin, hair, or semen are convenient sources of clinical samples. These sources are also suitable if the target is RNA. Blood and other body fluids are also a convenient source for isolating viral nucleic acids. If the target nucleic acid molecule is mRNA, the sample is obtained from a tissue in which the mRNA is expressed. If the target nucleic acid molecule in the sample is RNA, it may be reverse transcribed to DNA, but need not be converted to DNA in the present invention.

A plurality of collection methods can be used depending on the type of sample to be analyzed. Liquid samples can be collected by placing a constant volume of the liquid into a lysis buffer. Airborne samples can be collected by passing air over a filter for a constant time. The filter can be washed with lysis buffer. Alternatively, the filter can be placed directly into the lysis buffer. Waterborne samples can be collected by passing a constant amount of water over a filter. The filter can then be washed with lysis buffer or soaked directly in the lysis buffer. Dry samples can be directly deposited into lysis buffer for removal of the organism of interest.

When whole cells, viruses, or other tissue samples are being analyzed, it is typically necessary to extract the nucleic acids from the cells or viruses, prior to continuing with the various sample preparation operations. Accordingly, following sample collection, nucleic acids may be liberated from the collected cells, viral coat, etc., into a crude extract, followed by additional treatments to prepare the sample for subsequent operations such as denaturation of contaminating (DNA binding) proteins, purification, filtration, and desalting.

Liberation of nucleic acids from the sample cells or viruses, and denaturation of DNA binding proteins may generally be performed by physical or chemical methods. For example, chemical methods generally employ lysing agents to disrupt the cells and extract the nucleic acids from the cells, followed by treatment of the extract with chaotropic salts such as guanidinium isothiocyanate or urea, to denature any contaminating and potentially interfering proteins. Generally, where chemical extraction and/or denaturation methods are used, the appropriate reagents may be introduced into the fluid pathway of the detection card through the injection ports, the reagent reservoirs, or externally introduced.

Alternatively, physical methods may be used to extract the nucleic acids and denature DNA binding proteins. U.S. Pat. No. 5,304,487, which is hereby incorporated by reference in its entirety, discusses the use of physical protrusions within microchannels or sharp edged particles within a reservoir or channel to pierce cell membranes and extract their contents. More traditional methods of cell extraction may also be used, e.g., employing a channel with restricted cross-sectional dimension which causes cell lysis when the sample is passed through the channel with sufficient flow pressure. Alternatively, cell extraction and denaturing of contaminating proteins may be carried out by applying an alternating electrical current to the sample. More specifically, the sample of cells is flowed through a microtubular array while an alternating electric current is applied across the fluid flow. A variety of other methods may be utilized within the device of the present invention to effect cell lysis/extraction, including, e.g., subjecting cells to ultrasonic agitation, or forcing cells through microgeometry apertures, thereby subjecting the cells to high shear stress resulting in rupture.

Following extraction, it is often desirable to separate the nucleic acids from other elements of the crude extract, e.g., denatured proteins, cell membrane particles, and the like. Removal of particulate matter is generally accomplished by filtration, flocculation, or the like. Ideally, the sample is concentrated by filtration, which is more rapid and does not require special reagents. A variety of filter types may be readily incorporated into the detection card of the present invention. Samples can be forced through filters that will allow only the cellular material to pass through, trapping whole organisms and broken cell debris. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample. Suitable solid supports for nucleic acid binding include, e.g., diatomaceous earth, silica, or the like. Suitable gel exclusion media is also well known in the art and is commercially available from, e.g., Pharmacia and Sigma Chemical. This isolation and/or gel filtration/desalting may be carried out in an additional reservoir, or alternatively, the particular chromatographic media may be incorporated in a channel or fluid passage leading to a subsequent detection reservoir.

The probes are preferably selected to bind with the target such that they have approximately the same melting temperature. This can be done by varying the lengths of the hybridization region. A-T rich regions may have longer target sequences, whereas G-C rich regions would have shorter target sequences.

Hybridization assays on substrate-bound oligonucleotide arrays involve a hybridization step and a detection step. In the hybridization step, the sample potentially containing the target and an isostabilizing agent, denaturing agent, or renaturation accelerant is brought into contact with the probes of the array and incubated at a temperature and for a time appropriate to allow hybridization between the target and any complementary probes.

Including a hybridization optimizing agent in the hybridization mixture significantly improves signal discrimination between perfectly matched targets and single-base mismatches. As used herein, the term “hybridization optimizing agent” refers to a composition that decreases hybridization between mismatched nucleic acid molecules, i.e., nucleic acid molecules whose sequences are not exactly complementary.

An isostabilizing agent is a composition that reduces the base-pair composition dependence of DNA thermal melting transitions. More particularly, the term refers to compounds that, in proper concentration, result in a differential melting temperature of no more than about 1° C. for double stranded DNA oligonucleotides composed of AT or GC, respectively. Isostabilizing agents preferably are used at a concentration between 1 M and 10 M, more preferably between 2 M and 6 M, most preferably between 4 M and 6 M, between 4 M and 10 M, and, optimally, at about 5 M. For example, a 5 M agent in 2×SSPE (Sodium Chloride/Sodium Phosphate/EDTA solution) is suitable. Betaines and lower tetraalkyl ammonium salts are examples of suitable isostabilizing agents.

Betaine (N,N,N,-trimethylglycine; (Rees et al., Biochem. 32:137-144 (1993)), which is hereby incorporated by reference in its entirety) can eliminate the base pair composition dependence of DNA thermal stability. Unlike tetramethylammonium chloride (“TMACl”), betaine is zwitterionic at neutral pH and does not alter the polyelectrolyte behavior of nucleic acids while it does alter the composition-dependent stability of nucleic acids. Inclusion of betaine at about 5 M can lower the average hybridization signal, but increases the discrimination between matched and mismatched probes.

A denaturing agent is a composition that lowers the melting temperature of double stranded nucleic acid molecules by interfering with hydrogen bonding between bases in a double-stranded nucleic acid or the hydration of nucleic acid molecules. Denaturing agents can be included in hybridization buffers at concentrations of about 1 M to about 6 M and, preferably, about 3 M to about 5.5 M.

Denaturing agents include formamide, formaldehyde, dimethylsulfoxide (“DMSO”), tetraethyl acetate, urea, guanidine thiocyanate (“GuSCN”), glycerol and chaotropic salts. As used herein, the term “chaotropic salt” refers to salts that function to disrupt van der Waal's attractions between atoms in nucleic acid molecules. Chaotropic salts include, for example, sodium trifluoroacetate, sodium tricholoroacetate, sodium perchlorate, and potassium thiocyanate.

A renaturation accelerant is a compound that increases the speed of renaturation of nucleic acids by at least 100-fold. They generally have relatively unstructured polymeric domains that weakly associate with nucleic acid molecules. Accelerants include heterogenous nuclear ribonucleoprotein (“hnRP”) A1 and cationic detergents such as, preferably, cetyltrimethylammonium bromide (“CTAB”) and dodecyl trimethylammonium bromide (“DTAB”), and, also, polylysine, spermine, spermidine, single stranded binding protein (“SSB”), phage T4 gene 32 protein, and a mixture of ammonium acetate and ethanol. Renaturation accelerants can be included in hybridization mixtures at concentrations of about 1 μM to about 10 mM and, preferably, 1 μM to about 1 mM. The CTAB buffers work well at concentrations as low as 0.1 mM.

Addition of small amounts of ionic detergents (such as N-lauroyl-sarkosine) to the hybridization buffers can also be useful. LiCl is preferred to NaCl. Hybridization can be at 20°-65° C., usually 37° C. to 45° C. for probes of about 14 nucleotides. Additional examples of hybridization conditions are provided in several sources, including: Sambrook et al., Molecular Cloning: A Laboratory Manual, 2nd Ed., Cold Spring Harbor, N.Y. (1989); and Berger and Kimmel, “Guide to Molecular Cloning Techniques,” Methods in Enzymology, Volume 152, Academic Press, Inc., San Diego, Calif. (1987); Young and Davis, Proc. Natl. Acad. Sci. USA, 80:1194 (1983), which are hereby incorporated by reference in their entirety.

In addition to aqueous buffers, non-aqueous buffers may also be used. In particular, non-aqueous buffers which facilitate hybridization but have low electrical conductivity are preferred.

The sample and hybridization reagents are placed in contact with the detection chip and incubated in the detection reservoir of the detection card. Generally, incubation will be at temperatures normally used for hybridization of nucleic acids, for example, between about 20° C. and about 75° C., e.g., about 25° C., about 30° C., about 35° C., about 40° C., about 45° C., about 50° C., about 55° C., about 60° C., or about 65° C. For probes longer than about 14 nucleotides, 37-45° C. is preferred. For shorter probes, 55-65° C. is preferred. More specific hybridization conditions can be calculated using formulae for determining the melting point of the hybridized region. Preferably, hybridization is carried out at a temperature at or between ten degrees below the melting temperature and the melting temperature. More preferred, hybridization is carried out at a temperature at or between five degrees below the melting temperature and the melting temperature. The target is incubated with the capture probes for a time sufficient to allow the desired level of hybridization between the target and any complementary capture probes. After incubation with the hybridization mixture, the electrically separated conductors are washed with the hybridization buffer, which also can include the hybridization optimizing agent. These agents can be included in the same range of amounts as for the hybridization step, or they can be eliminated altogether.

Details on how capture probes are attached to electrical conductors are set forth in U.S. patent application Ser. Nos. 10/288,657 and 10/763,597, which are hereby incorporated by reference in their entirety.

Various other methods exist for attaching the capture probes to the electrical conductors. For example, U.S. Pat. Nos. 5,861,242, 5,856,174, 5,856,101, and 5,837,832, which are hereby incorporated by reference in their entirety, disclose a method where light is shone through a mask to activate functional (for oligonucleotides, typically an —OH) groups protected with a photo-removable protecting group on a surface of a solid support. After light activation, a nucleoside building block, itself protected with a photo-removable protecting group (at the 5′-OH), is coupled to the activated areas of the support. The process can be repeated, using different masks or mask orientations and building blocks, to place probes on a substrate.

Alternatively, new methods for the combinatorial chemical synthesis of peptide, polycarbamate, and oligonucleotide arrays have recently been reported (see Fodor et al., Science 251:767-773 (1991); Cho et al., Science 261:1303-1305 (1993); and Southern et al., Genomics 13:1008-10017 (1992), which are hereby incorporated by reference in their entirety). These arrays (see Fodor et al., Nature 364:555-556 (1993), which is hereby incorporated by reference in its entirety) harbor specific chemical compounds at precise locations in a high-density, information rich format, and are a powerful tool for the study of biological recognition processes.

Preferably, the probes are attached to the leads through spatially directed oligonucleotide synthesis. Spatially directed oligonucleotide synthesis may be carried out by any method of directing the synthesis of an oligonucleotide to a specific location on a substrate. Methods for spatially directed oligonucleotide synthesis include, without limitation, light-directed oligonucleotide synthesis, microlithography, application by ink jet, microchannel deposition to specific locations and sequestration with physical barriers. In general, these methods involve generating active sites, usually by removing protective groups, and coupling to the active site a nucleotide which, itself, optionally has a protected active site if further nucleotide coupling is desired.

In one embodiment, the lead-bound oligonucleotides are synthesized at specific locations by light-directed oligonucleotide synthesis which is disclosed in U.S. Pat. No. 5,143,854, WO 92/10092, and WO 90/15070, which are hereby incorporated by reference in their entirety. In a basic strategy of this process, the surface of a solid support modified with linkers and photolabile protecting groups is illuminated through a photolithographic mask, yielding reactive hydroxyl groups in the illuminated regions. A 3′-O-phosphoramidite-activated deoxynucleoside (protected at the 5′-hydroxyl with a photolabile group) is then presented to the surface and coupling occurs at sites that were exposed to light. Following the optional capping of unreacted active sites and oxidation, the substrate is rinsed and the surface is illuminated through a second mask, to expose additional hydroxyl groups for coupling to the linker. A second 5′-protected, 3′-O-phosphoramidite-activated deoxynucleoside (C—X) is presented to the surface. The selective photodeprotection and coupling cycles are repeated until the desired set of probes are obtained. Photolabile groups are then optionally removed, and the sequence is, thereafter, optionally capped. Side chain protective groups, if present, are also removed. Since photolithography is used, the process can be miniaturized to specifically target leads in high densities on the support.

The protective groups can, themselves, be photolabile. Alternatively, the protective groups can be labile under certain chemical conditions, e.g., acid. In this example, the surface of the solid support can contain a composition that generates acids upon exposure to light. Thus, exposure of a region of the substrate to light generates acids in that region that remove the protective groups in the exposed region. Also, the synthesis method can use 3′-protected 5′-O-phosphoramidite-activated deoxynucleoside. In this case, the oligonucleotide is synthesized in the 5′ to 3′ direction, which results in a free 5′ end.

The general process of removing protective groups by exposure to light, coupling nucleotides (optionally competent for further coupling) to the exposed active sites, and optionally capping unreacted sites is referred to herein as “light-directed nucleotide coupling.”

The probes may be targeted to the electrically separated conductors by using a chemical reaction for attaching the probe or nucleotide to the conductor which preferably binds the probe or nucleotide to the conductor rather than the support material. Alternatively, the probe or nucleotide may be targeted to the conductor by building up a charge on the conductor which electrostatically attracts the probe or nucleotide.

Nucleases can be used to remove probes which are attached to the wrong conductor. More particularly, a target nucleic acid molecule may be added to the probes. Targets which bind at both ends to probes, one end to each conductor, will have no free ends and will be resistant to exonuclease digestion. However, probes which are positioned so that the target cannot contact both conductors will be bound at only one end, leaving the molecule subject to digestion. Thus, improperly located probes can be removed while protecting the properly located probes. After the protease is removed or inactivated, the target nucleic acid molecule can be removed and the device is ready for use.

The capture probes can be formed from natural nucleotides, chemically modified nucleotides, or nucleotide analogs, as long as they have activated hydroxyl groups compatible with the linking chemistry. Such RNA or DNA analogs comprise but are not limited to 2′-O-alkyl sugar modifications, methylphosphonate, phosphorothioate, phosphorodithioate, formacetal, 3′-thioformacetal, sulfone, sulfamate, and nitroxide backbone modifications, amides, and analogs, where the base moieties have been modified. In addition, analogs of oligomers may be polymers in which the sugar moiety has been modified or replaced by another suitable moiety, resulting in polymers which include, but are not limited to, polyvinyl backbones (Pitha et al., “Preparation and Properties of Poly (I-vinylcytosine),” Biochim. Biophys. Acta 204:381-8 (1970); Pitha et al., “Poly(1-vinyluracil): The Preparation and Interactions with Adenosine Derivatives,” Biochim. Biophys. Acta 204:39-48 (1970), which are hereby incorporated by reference in their entirety), morpholino backbones (Summerton, et al., “Morpholino Antisense Oligomers: Design, Preparation, and Properties,” Antisense Nucleic Acid Drug Dev. 7:187-9 (1997), which is hereby incorporated by reference in its entirety) and peptide nucleic acid (PNA) analogs (Stein et al., “A Specificity Comparison of Four Antisense Types: Morpholino, 2′-O-methyl RNA, DNA, and Phosphorothioate DNA,” J. Antisense Nucleic Acid Drug Dev. 7:151-7 (1997); Faruqi et al., “Peptide Nucleic Acid-Targeted Mutagenesis of a Chromosomal Gene in Mouse Cells,” Proc. Natl. Acad. Sci. USA 95:1398-403 (1998); Christensen et al., “Solid-Phase Synthesis of Peptide Nucleic Acids,” J. Pept. Sci. 1:175-83 (1995); Nielsen et al., “Peptide Nucleic Acid (PNA). A DNA Mimic with a Peptide Backbone,” Bioconjug. Chem. 5:3-7 (1994), which are hereby incorporated by reference in their entirety).

The capture probes can contain the following exemplary modifications: pendant moieties, such as, proteins (including, for example, nucleases, toxins, antibodies, signal peptides and poly-L-lysine); intercalators (e.g., acridine and psoralen), chelators (e.g., metals, radioactive metals, boron and oxidative metals), alkylators, and other modified linkages (e.g., alpha anomeric nucleic acids). Such analogs include various combinations of the above-mentioned modifications involving linkage groups and/or structural modifications of the sugar or base for the purpose of improving RNAseH-mediated destruction of the targeted RNA, binding affinity, nuclease resistance, and or target specificity.

The present invention can be used for numerous applications, such as detection of pathogens. For example, samples may be isolated from drinking water or food and rapidly screened for infectious organisms. The present invention may also be used for food and water testing. In recent times, there have been several large recalls of tainted meat products. The detection system of the present invention can be used for the in-process detection of pathogens in foods and the subsequent disposal of the contaminated materials. This could significantly improve food safety, prevent food borne illnesses and death, and avoid costly recalls. Capture probes that can identify common food borne pathogens, such as Salmonella and E. coli, could be designed for use within the food industry.

In yet another embodiment, the present invention can be used for real time detection of biological warfare agents. With the recent concerns of the use of biological weapons in a theater of war and in terrorist attacks, the device could be configured into a personal sensor for the combat soldier or into a remote sensor for advanced warnings of a biological threat. The devices which can be used to specifically identify the agent, can be coupled with a modem to send the information to another location. Mobile devices may also include a global positioning system to provide both location and pathogen information.

In yet another embodiment, the present invention may be used to identify an individual. A series of probes, of sufficient number to distinguish individuals with a high degree of reliability, are placed within the device. Various polymorphism sites are used. Preferentially, the device can determine the identity to a specificity of greater than one in one million, more preferred is a specificity of greater than one in one billion, even more preferred is a specificity of greater than one in ten billion.

### EXAMPLES

The following examples are provided to illustrate embodiments of the present invention but are by no means intended to limit its scope.

### Example 1

Detection of Target Nucleic Acid Molecules in a Sample Containing Purified DNA

In a prophetic example, a 10 μl sample containing approximately 100 ng of purified DNA dissolved in hybridization buffer (100 mM NaPhosphate, pH 7.5, 0.1% SDS) with a defined length of 5.7 kilobases is injected into the archival reservoir. The nucleic acid denatures for approximately 1 minute before the reservoir is evacuated and the sample passed along to the detection reservoir. The nucleic acid sample resides in the detection reservoir over the test structures for 5 minutes at a temperature of 55° C. The sample is evacuated from the detection reservoir with a 10 sample volume wash with hybridization buffer. The nucleic acid sample is washed into the waste reservoir. A 10 sample volume wash with distilled and deionized water rinses out the reservoir and prepares the sensor for chemical coating. The metallization chemistry is then mixed on a card having electrically separated conductors and passed through the detection reservoir at a fixed flow rate such that the test structures are in contact with the solution for a defined time. The test structures are rinsed with 10 sample volumes of distilled and deionized water. The test structures are then electrically probed individually to determine the resistance of each test structure. Resistance is obtained by applying a voltage between the two electrical test pads and measuring the associated current. The resistance is calculated from Ohm's Law. Low resistance indicates the metallization process has fused two electrodes and is a positive result.

### Example 2

Detection of Target Nucleic Acid Molecules in a Sample Containing Bacteria

In a prophetic example, a known quantity of bacteria are placed into lysis solution (Tris-CL, SDS) for 1 minute to break open bacteria. The cell debris is removed via filtration and the genomic DNA sheared by passing the solution through a point-sink shearing cartridge (65 μm diameter tubing). A 10 μl sample of the partially purified lysate in hybridization buffer (100 mM NaPhosphate, pH 7.5, 0.1% SDS) is injected into the archival reservoir. The nucleic acid denatures for approximately 1 minute before the reservoir is evacuated and the sample is passed along to the detection reservoir. The nucleic acid sample resides in the detection reservoir over the test structures for 5 minutes at a temperature of 55 degrees. The sample is evacuated from the detection reservoir with a 10 sample volume wash with hybridization buffer. The nucleic acid sample is washed into the waste reservoir. A 10 sample volume wash with distilled and deionized water rinses out the reservoir and prepares the sensor for chemical coating. The metallization chemistry is then mixed on a card having electrically separated conductors and passed through the detection reservoir at a fixed flow rate such that the test structures are in contact with the solution for a defined time. The test structures are rinsed with 10 sample volumes of distilled and deionized water. The test structures are then electrically probed individually to determine the resistance of each test structure. Resistance is obtained by passing a current (200 nA) through one of the two electrical test pads on each test structure and measuring the resistance between the two electrodes. Low resistance indicates the metallization process has fused two electrodes and is a positive result.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A device suitable for detecting a target molecule in a sample using a detection card having two or more electrically separated conductors, wherein the sample can be analyzed for the presence of the target molecule by determining whether the conductors are electrically connected, said device comprising:
a platform for receiving said detection card;
a lid moveable from a first position in engagement with the platform to a second position distal from the platform, wherein the lid comprises:
a fluid interface suitable for establishing fluid communication between the device and the detection card when the lid is in the first position, but not the second position, and the detection card is positioned on the platform and
an electrical interface suitable for establishing electrical communication between the device and the detection card when the lid is in the first position, but not the second position, and the detection card is positioned on the platform.

2. The device according to claim 1, wherein the lid is pivotally moveable between the first and second positions.

3. The device according to claim 1, wherein the platform comprises one or more alignment pins which correspond to one or more holes in the detection card to position the detection card correctly on the platform.

4. The device according to claim 1, wherein the platform comprises a heat exchanger to control fluid temperature in the detection card when positioned on the platform.

5. The device according to claim 1 further comprising:
a fluidic system connected to the fluid interface, wherein the fluidic system comprises:
one or more vials;
one or more fluid lines connecting the one or more vials to the fluid interface; and
one or more fluid displacement devices to move fluid from the one or more vials through the one or more fluid lines to the fluid interface.

6. The device according to claim 5, wherein the fluidic system further comprises:
one or more solenoid valves positioned in the fluid lines in a configuration suitable to oscillate fluid within the detection card.

7. The device according to claim 5, wherein the one or more vials comprise:
a vial carrying water;
a vial carrying metal solution;
a vial carrying a first developer solution;
a vial carrying a second developer solution; and
a vial carrying buffer solution.

8. The device according to claim 1 further comprising:
a controller electrically connected to the electrical interface to establish electrical communication between the electrically separated conductors of the detection card and the controller when the lid is in the first position, but not the second position, whereby a presence of the target molecule in the sample can be detected by the detection card and the device collectively.

9. The device according to claim 8 further comprising:
an external computer electrically coupled to the controller.

10. The device according to claim 9, wherein the external computer is a desk-top unit.

11. The device according to claim 9, wherein the external computer is a handheld computing device.

12. A system for detecting a target molecule in a sample, said system comprising:
a detection card comprising:
one or more pairs of electrical conductors, each pair including a first electrical conductor and a second electrical conductor, wherein the electrical conductors are not in contact with one another and
one or more sets of two oligonucleotide probes attached to the electrical conductors, wherein the probes are positioned such that they cannot come into contact with one another and such that a target nucleic acid molecule, which has two sequences, a first sequence complementary to a first probe attached to the first electrical conductor and a second sequence complementary to a second probe attached to the second electrical conductor, can bind to both probes and
a device suitable for receiving the detection card, said device comprising:
a platform for receiving said detection card;
a lid moveable from a first position in engagement with the platform to a second position distal from the platform, wherein the lid comprises:
a fluid interface suitable for establishing fluid communication between the device and the detection card when the lid is in the first position, but not the second position, and the detection card is positioned on the platform and
an electrical interface suitable for establishing electrical communication between the device and the detection card when the lid is in the first position, but not the second position, and the detection card is positioned on the platform.

13. The system according to claim 12, wherein the two or more electrically separated conductors are in the form of spaced apart conductive fingers.

14. The system according to claim 12, wherein a plurality of pairs of spaced apart conductive fingers are present in the detection card.

15. The system according to claim 12, wherein the capture probes are oligonucleotides.

16. The system according to claim 12, wherein the capture probes are peptide nucleic acid analogs.

17. The system according to claim 12, wherein the lid is pivotally moveable between the first and second position.

18. The system according to claim 12, wherein the detection card further comprises:
a detection chip comprising a substrate upon which the electrical conductors are fabricated.

19. The system according to claim 18, wherein the detection card comprises a fluid pathway having a detection reservoir into which at least part of a detection chip is received.

20. The system according to claim 19, wherein the fluid pathway further comprises:
a sample injection port through which a sample can be introduced into the detection reservoir.

21. The system according to claim 19, wherein the detection card further comprises:
electrical contacts extending through the detection card and coupled to the electrically separated conductors of the detection chip.

22. The system according to claim 21, wherein the detection card further comprises:
a plurality of injection ports through which reagents can be introduced into the detection reservoir.

23. A method of detecting a target molecule in a sample, said method comprising:
providing a detection system comprising:
a detection card comprising:
one or more pairs of electrical conductors, each pair including a first electrical conductor and a second electrical conductor, wherein the electrical conductors are not in contact with one another and
one or more sets of two oligonucleotide probes attached to the electrical conductors, wherein the probes are positioned such that they cannot come into contact with one another and such that a target nucleic acid molecule, which has two sequences, a first sequence complementary to a first probe attached to the first electrical conductor and a second sequence complementary to a second probe attached to the second electrical conductor, can bind to both probes;
a sample injection port through which a sample can be introduced into the detection card; and
a device suitable for receiving the detection card, said device comprising:
a platform for receiving said detection card;
a lid moveable from a first position in engagement with the platform to a second position distal from the platform, wherein the lid comprises:
a fluid interface suitable for establishing fluid communication between the device and the detection card when the lid is in the first position, but not the second position, and the detection card is positioned on the platform and
an electrical interface suitable for establishing electrical communication between the device and the detection card when the lid is in the first position, but not the second position, and the detection card is positioned on the platform;
injecting a sample, potentially containing the target molecule, into the sample injection port;
positioning the detection card on the platform with the lid in the second position;
moving the lid into the first position such that fluid communication and electrical communication is established between the device and the detection card by the fluid interface and the electrical interface, respectively;
processing the sample within the detection card under conditions effective to permit any of the target molecule present in the sample to bind to the capture probes and thereby connect the capture probes; and
detecting the presence of the target molecule by determining whether electricity is conducted between the electrically separated conductors.

24. The method according to claim 23, wherein the target molecule is selected from the group consisting of DNA, RNA, chemically modified nucleic acid molecules, and nucleic acid analogs.

25. The method according to claim 23, wherein the sample is saliva, whole blood, peripheral blood lymphocytes, skin, hair, or semen.

26. The method according to claim 23, wherein said method is used to detect infectious agents.

27. The method according to claim 23, wherein said method is used for nucleic acid sequencing.

28. The method according to claim 23, wherein one or both of the probes has a sequence which is complementary to a sequence having a polymorphism, wherein the base or bases complementary to the polymorphism are located at an end of the probe distal to the conductors.

29. The method according to claim 23, wherein said processing comprises:
neutralizing the sample;
contacting the neutralized sample with a buffer;
treating the sample with a conductive ion solution after said contacting with a buffer; and
treating the sample with an enhancer after said treating with a conductive ion solution.

30. The method according to claim 23 further comprising:
coating any target molecules bound to the capture probes with a conductive ion material after said processing and before said detecting.
